(19) United States
(12) Patent Application Publication
(10) Pub. No.: US 2014/0221819 A1
(43) Pub. Date: Aug. 7, 2014

(54) APPARATUS, SYSTEM AND METHOD FOR SURGICAL NAVIGATION

(71) Applicant: David SARMENT, Burke, VA (US)

(72) Inventor: David SARMENT, Burke, VA (US)

(21) Appl. No.: 13/757,490

(22) Filed: Feb. 1, 2013

(51) Int. Cl. A61B 5/06; A61B 1/24; A61B 5/00

(52) U.S. Cl. CPC A61B 5/064; A61B 5/0077; A61B 1/24; A61B 5/7445; A61B 5/72
USPC 600/424

(57) ABSTRACT

A surgical navigation system. The surgical navigation system can include a computing device having a non-transitory computer-readable medium for storing surgical navigation software, and a processor adapted to execute the software, a headgear unit communicatively coupled to the computing device, the headgear unit including at least one camera. The system can further include at least one fiducial marker, the fiducial marker having a complex configuration, wherein the configuration of the fiducial marker is adapted to present a unique view of the fiducial marker to the at least one camera based on the angle between the camera and the fiducial marker, and wherein the surgical navigation software is adapted to determine the position of the fiducial marker in three-dimensional space based on the unique view of the fiducial marker.

### BACKGROUND

Surgical navigation, also known as computer-aided surgery, involves the use of computer technology for aiding surgical operations. A typical workflow for surgical navigation applications can include: the acquisition of the diagnostic image utilizing a guide with fiducial markers; surgical planning via software; patient preparation by repositioning or verifying the position of the guide with fiducial markers; calibration of the equipment to verify that the guide and fiducial markers are recorded properly and accurately; and the surgery itself.

Acquisition of the diagnostic image involves obtaining an accurate model of the region of the patient where the surgery is to be performed. Typically, this is performed by imaging the patient via one of several methods, including computed tomography (CT), x-rays, ultrasound, and the like. In the CT method, a guide having a radiopaque fiducial marker is typically positioned and secured to a repeatable location in the region to be scanned. An image of the region is then obtained, for example by a CT scan, and/or a laser scan. The obtained images are then studied to plan the surgical approach. At the time of surgery, the guide is repositioned at the location and equipped with fiducial markers. The surgical instruments to be used are likewise equipped with fiducial markers. Both sets of tracking devices are visible to an optical tracking system in the surgical operation area. The optical tracking system typically includes a plurality of cameras or other imaging devices, allowing the system to determine the location of the fiducial markers in three dimensions. As the fiducial markers are at a known location with respect to the image data, the surgical navigation software can quickly and easily determine the position of the patient anatomy and the position of the surgical instruments in relation thereto.

In the dentistry field, surgical navigation has limited but increasing penetration. For example, a dental surgeon can use a CT scan to plan for surgical placement of dental implants. Such implants are typically cylindrical or conical screw-like metal inserts having a platform end and an apex end. The apex end is inserted into an aperture formed in the jaw bone, while the platform end is used for coupling a dental crown or other prosthesis. As the implant location within the bone and the angle of the implant are important to satisfactory positioning of the implant, as well as to avoid critical anatomical features such as nerves, the implant can be provided with a fiducial marker. A fiducial marker can be provided at another location, such as a tooth next to the implant location, another implant, the longitudinal axis of a crown to be placed on the implant, a fixed object, or any other location. The angle and distance between the two fiducial markers can then be measured.

Several difficulties are present in surgical navigation. The calibration of the equipment is typically time consuming and difficult due to the precision required. The calibration may be lost during the surgery, requiring that the surgical procedure be stopped to reestablish calibration. Furthermore, the quality of the calibration itself can be difficult to measure, while being critical to precision.

Additionally, the surgical procedure requires an uninterrupted line of sight between the fiducial markers and the surgical navigation optical tracking system. During surgery, the positions of the individuals and instruments involved may need to be altered so as to gain optimal access to the surgical site. The uninterrupted line of sight can thus be difficult to maintain, and can frequently be lost, for example when objects or people are positioned between the cameras of the tracking system and the fiducial markers. Consequently, when line of sight is lost, the surgical navigation procedure is interrupted.

### SUMMARY

According to at least one exemplary embodiment, a surgical navigation system is disclosed. The surgical navigation system can include a computing device having a non-transitory computer-readable medium for storing surgical navigation software, and a processor adapted to execute the software, a headgear unit communicatively coupled to the computing device, the headgear unit including at least one camera. The system can further include at least one fiducial marker, the fiducial marker having a complex configuration, wherein the configuration of the fiducial marker is adapted to present a unique view of the fiducial marker to the at least one camera based on the angle between the camera and the fiducial marker, and wherein the surgical navigation software is adapted to determine the position of the fiducial marker in three-dimensional space based on the unique view of the fiducial marker. The field of view of the camera can substantially coincide with a portion of the field of view of a user.

According to another exemplary embodiment, a method for surgical navigation is disclosed. The method can include providing a headgear device including at least one camera, the headgear device adapted to be worn by a user, providing at least one fiducial marker having a complex configuration, and determining the position and orientation of the at least one fiducial marker based on the spatial relationship between the at least one fiducial marker and the at least one camera, wherein a field of view of the at least one camera substantially coincides with portion of a field of view of the user. The method can further include providing at least one display device on the headgear device, and displaying surgical navigation images to the user via the at least one display device.

### BRIEF DESCRIPTION OF THE FIGURES

Advantages of embodiments of the present invention will be apparent from the following detailed description of the exemplary embodiments. The following detailed description should be considered in conjunction with the accompanying figures in which:

FIG. 1 is a schematic of an exemplary embodiment of a system for surgical navigation.

FIG. 2 is a schematic of an exemplary embodiment of headgear for use with a system for surgical navigation.

FIGS. 3a-3c shows exemplary embodiments of grids for fiducial marker for use with a system for surgical navigation.

FIGS. 4a-4d show exemplary embodiments of guides and instruments on which the fiducial markers may be used.

### DETAILED DESCRIPTION

Aspects of the invention are disclosed in the following description and related drawings directed to specific embodiments of the invention. Alternate embodiments may be devised without departing from the spirit or the scope of the invention. Additionally, well-known elements of exemplary embodiments of the invention will not be described in detail or will be omitted so as not to obscure the relevant details of the invention. Further, to facilitate an understanding of the description discussion of several terms used herein follows.

As used herein, the word “exemplary” means “serving as an example, instance or illustration.” The embodiments described herein are not limiting, but rather are exemplary only. It should be understood that the described embodiment are not necessarily to be construed as preferred or advantageous over other embodiments. Moreover, the terms “embodiments of the invention”, “embodiments” or “invention” do not require that all embodiments of the invention include the discussed feature, advantage or mode of operation.

Further, many of the embodiments described herein are described in terms of sequences of actions to be performed by, for example, elements of a computing device. It should be recognized by those skilled in the art that the various sequence of actions described herein can be performed by specific circuits (e.g., application specific integrated circuits (ASICs)) and/or by program instructions executed by at least one processor. Additionally, the sequence of actions described herein can be embodied entirely within any form of non-transitory computer-readable storage medium such that execution of the sequence of actions enables the processor to perform the functionality described herein. Thus, the various aspects of the present invention may be embodied in a number of different forms, all of which have been contemplated to be within the scope of the claimed subject matter. In addition, for each of the embodiments described herein, the corresponding form of any such embodiments may be described herein as, for example, “a computer configured to” perform the described action.

According to at least one exemplary embodiment, an apparatus, system and method for surgical navigation is disclosed. The embodiments disclosed herein can include fiducial markers having complex radiopaque marking grids thereon. The radiopaque marking grids of the fiducial markers may have complex configurations and may have three-dimensional shapes. The fiducial markers may be provided separately, as part of surgical navigation guides, and/or as part of surgical instruments. Exemplary embodiments of the complex radiopaque marking grids can include patterns or grids of triangles, circles, or other shapes. The surfaces and/or marking grids of the fiducial markers may be configured such that the image of the fiducial marker captured by an optical tracking system camera can substantially differ based on the spatial relationship between the camera and the fiducial marker, thereby facilitating the determination of the position of the fiduciary marker in three-dimensional space. In other words, the image of the fiducial marker captured by the optical tracking system camera can be substantially unique based on the relative locations and orientations of the marker and the camera in three-dimensional space; for example, the distance between the marker and the camera, the viewing angle between the marker and the camera, the particular surface or surfaces of the marker that can be viewed by the camera, the orientation and/or angle of such surfaces with respect to the camera, and so forth. Consequently, the need for calibration and re-calibration of the surgical navigation equipment may be significantly reduced.

The embodiments disclosed herein can further include a headgear device including cameras or other imaging devices disposed thereon. The cameras can be oriented to have a field of vision that is substantially within the field of view of a surgeon executing the surgical procedure, and can be in communication with the optical tracking system of the surgical navigation system. The cameras can therefore facilitate providing a substantially uninterrupted line-of-sight to the surgical site and the fiducial markers in the surgical site. In some exemplary embodiments, the headgear device can further include a display screen, allowing the surgeon to see visual output of a surgical navigation system. The field of view of the cameras may be within the field of view of the surgeon, may substantially overlap the field of view of the surgeon, may contain the field of view of the surgeon, or may coincide with the field of view of the surgeon. In other words, the cameras may be configured such that the scene observed by the cameras substantially corresponds to the scene observed by the surgeon wearing the headgear device.

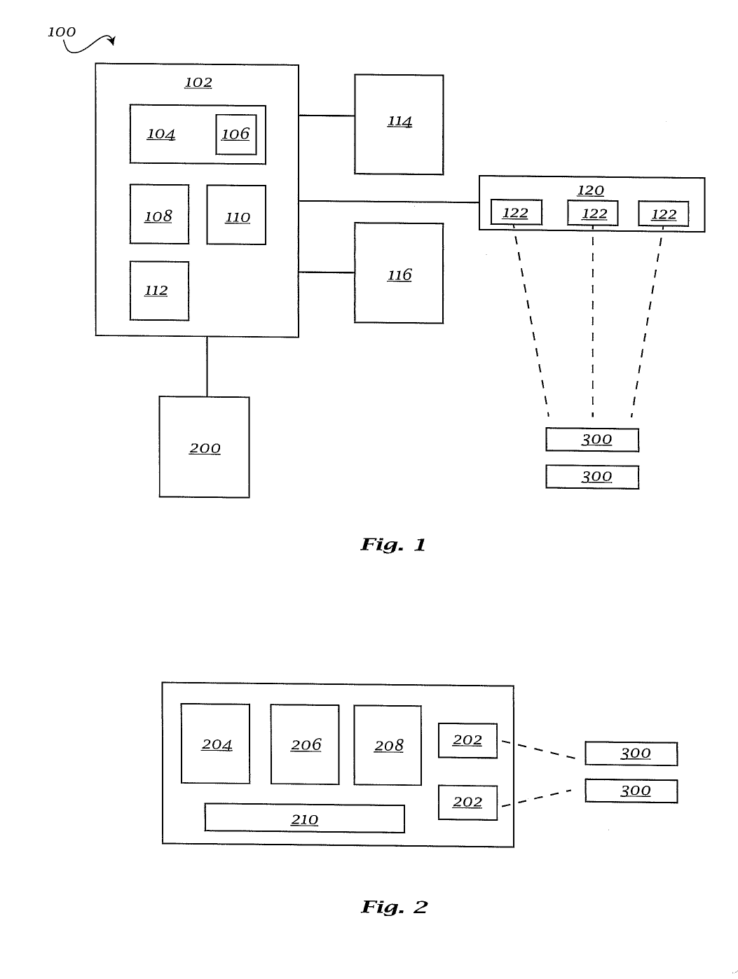

FIG. 1 shows an exemplary embodiment of a surgical navigation system 100. System 100 may include a computing device 102 having a non-transitory computer-readable medium 104 on which a surgical navigation software 106 may be stored, a memory 108, a processor 110 for executing the surgical navigation software 106, and a wireless communication device 112. Computing device 102 may be coupled to a display device 114, and may include at least one input device 116, such as a mouse, keyboard, touch-sensitive device, or the like.

System 100 can further include an optical tracking device 120 and may be communicatively coupled thereto. The optical tracking device 120 may be adapted to track a plurality of fiducial markers. Optical tracking device 120 can include a plurality of cameras 122 arranged in any desired configuration. Output from optical tracking device 120 may be processed by surgical navigation software 106 substantially as known to one having ordinary skill in the art.

System 100 can further include headgear 200, which is shown in FIG. 2. Headgear 200 can be adapted to be worn by a person, for example a surgeon taking part in the computer-aided surgery procedure. To that end, headgear 200 can include structures adapted to couple headgear 200 to the head of the wearer. In some exemplary embodiments, headgear 200 may be provided as a headband, as eyewear, or any other apparatus that enables headgear 200 to function as described herein. Headgear 200 can further include at least one camera 202 disposed thereon. Camera 202 may be oriented such that the field of view of camera 202 is oriented in substantially the same direction as the field of view of the person wearing headgear 200. Camera 202 may be positioned such that the vantage point of camera 202 is proximate the vantage point of the user. Furthermore, camera 202 may be configured such that the field of view of camera 202 substantially coincides with the field of view of the user.

Headgear 202 may further be provided with a processor 204, power source 206 and a wireless communications device 208. Power source 206 may be any known power source, for example a rechargeable battery. Wireless communication device 208 can facilitate transmitting data between headgear 200 and computing device 102.

Headgear 200 can further include at least one display device 210. Display device may be positioned such that the device is in the field of view of the user of headgear 200 and such that the image displayed on device 210 is viewable by the user. Display device 210 may be any known display device that enables headgear 200 to function as disclosed herein. In some exemplary embodiments, display device 200 may be a liquid-crystal display screen positioned at a distance from the eyes of the user. In other exemplary embodiments, display device 200 may include a transparent portion disposed in front of the eye of the wearer, with a image overlay displayed thereon.

In some exemplary embodiments, display device 210 may display a surgical navigation image as commonly known in the art. For example, display device 210 may mirror the video output of system 100 that is displayed on display device 114. In further exemplary embodiments, display device 210 may display a view that corresponds to the field of view, or a portion of the field of view, of the wearer of display device 210. In further exemplary embodiments, display device 210 may provide an image overlay that can correspond to the view through the transparent portion of display device 200. For example, the image overlay may be provided on a transparent portion of the display device in the manner commonly known as “augmented reality.”

It should be appreciated that system 100 may be used without optical tracking device 120. In such embodiments, headgear 200 and the cameras 202 therein may provide the tracking functionality ordinarily provided by tracking device 120, and may serve as the primary optical tracking device. If desired, optical tracking device 120 may be used as a supplement or as a redundant tracking device to headgear 202.

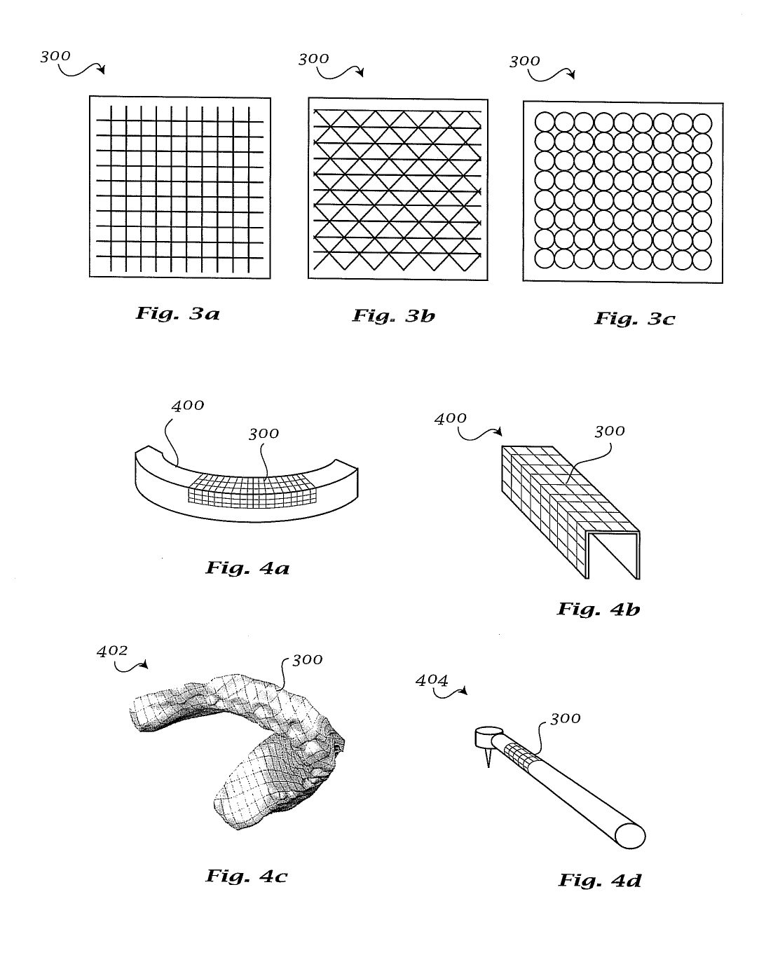

System 100 can further include a plurality of fiducial markers 300, as shown in FIGS. 3a-3c. Fiducial markers 300 may be formed from any radiopaque material, and can be configured as a complex grid. Exemplary configurations for the fiducial markers can include a grid, an arrangement or pattern of rectangles, triangles, circles, or other geometric shapes, or any other pattern or shape arrangement that enables system 100 to function as described herein.

As shown in FIGS. 4a-4c, fiducial markers 300 may be applied to a guide that may be positioned in the region to be scanned. One exemplary embodiment of a guide 400 may be made of a substantially rigid material, and may be configured to fit in or on a desired part of the patient anatomy. Another exemplary embodiment of a guide 402 may be made of a semi-rigid or pliable material, allowing guide 402 to be deformed so as to conform to the desired part of the patient anatomy. For example, a guide 402 may have a shape, or may be shaped or deformed, to fit in a dental arch of the patient or to fit on another anatomical landmark that is visible during surgery. The specific materials used for guides 400, 402 may be any suitable materials known to one having ordinary skill in the art.

As shown in FIGS. 4a-4c, exemplary embodiments of guides 400, 402 for use with system 100 may have three-dimensional shapes or surfaces. A fiducial marker 300 may be applied to a guide such that the grid, pattern, or shape arrangement of the fiducial marker 300 conforms to the three-dimensional shape or surface of the guide. As a result, the grid of the fiducial marker 300 can have a three-dimensional shape. Since the projected image of a fiducial marker 300, as viewed by a camera of system 100, is unique based on the spatial relationship between the marker and the camera, the position and orientation of the fiducial marker 300 can therefore be easily identified by the cameras of system 100.

Yet other fiducial markers included in system 300 may be applied to one or more instruments 404 to be used in the surgical operation, as shown in FIG. 4d. The fiducial markers 300 may be applied to any portion of the instrument 404 that enables system 100 to function as described herein. For example, the fiducial marker 300 may be positioned proximate the operating end of the surgical instrument 404, between the operating end and the handle portion, or in any other desired location on the surgical instrument 404. If desired, the fiducial marker 300 may be applied to surgical instrument 404 such that the grid, pattern, or shape arrangement of the fiducial marker 300 conforms to the three-dimensional shape or surface of the surgical instrument 404.

In operation, the fiducial markers 300 that are being used for a particular surgical operation may be imaged from a plurality of diverse vantage points, with each vantage point being a particular spatial relationship between the fiducial marker 300 and a camera 122 or 202. Such imaging can provide software 106 with imaging data that can allow software 106 to analyze the configuration and three-dimensional shape of the grid of the fiducial marker 300, and can allow software 106 to obtain images of the fiducial marker that correspond to particular spatial relationships between fiducial marker 300 and a camera 122 or 202. Furthermore, based on the imaging data, software 106 can further determine, for example by interpolation, additional views of the fiducial marker that would correspond to additional spatial relationships between fiducial marker 300 and a camera 122 or 202. In this manner, software 106 can construct a model of the fiducial marker 300 and correlate a plurality of views of the marker to corresponding spatial relationships between the marker and a camera 122 or 102.

Subsequently, at least one fiducial marker 300 may be placed in a desired location in the area to be operated upon, and a diagnostic image can be acquired. For example, in a dental application, at least one fiducial marker may be positioned on a tooth of the patient, on the jawbone, in a dental arch, on an implant, or at any desired location. However, the use of system 100 for surgical navigation in any other type of surgery may be contemplated and provided as desired. Surgical planning utilizing software 106 may then be performed according to known methods.

Prior to surgery, the fiducial markers may be replaced in their original positions. Other fiducial markers may be provided on any surgical tools to be used during the procedure. As the fiducial markers used with system 100 may have a variety of complex shapes, substantially as described above, the necessity to calibrate of the surgical equipment may be minimized. Similarly, the necessity to recalibrate occurring during the procedure may likewise be minimized. As the surfaces and grids of the fiducial markers are complex, the view of a fiducial marker as seen by any one of cameras 122 or 206 may be unique, depending on the spatial relationship between the fiducial marker and the particular camera. Software 106 can therefore analyze the captured image of the surface and grid of the fiducial marker, and consequently determine the location in space and the orientation of the particular fiducial marker. Once the position of the fiducial marker in space is known, the imagery of the patient anatomy may then be retrieved. Similarly, for a fiducial marker on a surgical instrument, software 106 can analyze the captured image of the surface and grid of the fiducial marker of the surgical instrument, and consequently determine the location in space and the orientation of the surgical instrument in relation to the patient anatomy. The image analysis and subsequent location and orientation determination may be performed by any software known in the art that enables system 100 to function as described herein, for example, software adapted for object shape detection and recognition.

Furthermore, in operation, system 100 may be used with one or both of imaging device 120 and headgear 200. While imaging device 120 can provide a field of view of the operating area as known in the art, cameras 206 of headgear 200 can provide a field of view that is substantially similar to the field of view of the surgeon wearing headgear 200. The field of view of cameras 206 may be substantially the same as the field of view of the surgeon, or may be larger or smaller than the field of view of the surgeon. Due to the correspondence between the field of view of the surgeon and the field of view of cameras 206, line of sight obstructions between cameras 206 and the fiducial markers can therefore be minimized, thereby streamlining the surgical navigation process.

Furthermore, in operation, system 100 may be used with one or both of display device 114 and headgear 200. The images generated by software 104 may thus be displayed on one or both display device 114 and display device 210 of headgear 200. When the display device 210 of headgear 200 is used for displaying the surgical navigation images, the surgeon may observe the surgical navigation images on the display device 210. This can allow the surgeon to observe the surgical navigation images while maintaining the operating area in the surgeon's field of view, thereby further streamlining the surgical navigation process and reducing the likelihood of error.

While the embodiments of the surgical navigation system, apparatus and method disclosed herein have been described in relation to the dentistry field, it should be appreciated that these embodiments may be utilized for any medical application where surgical navigation is used. Therefore, the embodiments described herein should not be construed as limited solely to dentistry.

The foregoing description and accompanying figures illustrate the principles, preferred embodiments and modes of operation of the invention. However, the invention should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments can be made by those skilled in the art without departing from the scope of the invention as defined by the following claims.

What is claimed is:

1. A surgical navigation system, comprising:
a computing device having a non-transitory computer-readable medium for storing surgical navigation software, and a processor adapted to execute the software;
a headgear unit communicatively coupled to the computing device, the headgear unit including at least one camera; and
at least one fiducial marker, the fiducial marker having a complex configuration;
wherein the configuration of the fiducial marker is adapted to present a unique view of the fiducial marker to the at least one camera based on the spatial relationship between the camera and the fiducial marker;
wherein the surgical navigation software is adapted to determine the position of the fiducial marker in three-dimensional space based on the unique view of the fiducial marker.

2. The surgical navigation system of claim 1, wherein headgear unit further comprises at least one display screen viewable by a wearer of the headgear unit.

3. The surgical navigation system of claim 1, wherein the complex configuration of the fiducial marker comprises a three-dimensional shape.

4. The surgical navigation system of claim 1, wherein the complex configuration of the fiducial marker comprises a complex radiopaque grid.

5. The surgical navigation system of claim 4, wherein the complex radiopaque grid comprises an arrangement of circles.

6. The surgical navigation system of claim 4, wherein the complex radiopaque grid comprises an arrangement of triangles.

7. The surgical navigation system of claim 1, wherein the fiducial marker is applied to a guide, the guide being configured so as to couple to a portion of a portion of the anatomy of a patient.

8. The surgical navigation system of claim 7, wherein the guide is adapted to conform to a portion of the patient anatomy.

9. The surgical navigation system of claim 1, wherein the fiducial marker is applied to a surgical instrument.

10. A surgical navigation system, comprising:
a headgear unit adapted to be worn by a user performing a surgical operation;
at least one camera coupled to the headgear unit;
at least one video display device coupled to the headgear unit and adapted to be viewable by the user;
wherein a field of view of the at least one camera substantially coincides with portion of a field of view of the user.

11. The surgical navigation system of claim 10, wherein the vantage point of the at least one camera is proximate the vantage point of the user.

12. The surgical navigation system of claim 10, further comprising:
at least one fiducial marker, wherein the configuration of the fiducial marker is adapted to present a unique view of the fiducial marker to the at least one camera based on the spatial relationship between the camera and the fiducial marker.

13. The surgical navigation system of claim 10, wherein the complex configuration of the fiducial marker comprises a complex radiopaque grid.

14. The surgical navigation system of claim 10, wherein the fiducial marker is applied to a guide, the guide being configured so as to couple to a portion of a portion of the anatomy of a patient.

15. The surgical navigation system of claim 10, wherein the fiducial marker is applied to a surgical instrument.

16. A method for surgical navigation, comprising:
providing a headgear device including at least one camera, the headgear device adapted to be worn by a user;
providing at least one fiducial marker having a complex configuration; and
determining the position and orientation of the at least one fiducial marker based on the spatial relationship between the at least one fiducial marker and the at least one camera;
wherein a field of view of the at least one camera substantially coincides with portion of a field of view of the user.

17. The method of claim 16, further comprising:
providing at least one display device on the headgear device; and
displaying surgical navigation images to the user via the at least one display device.

18. The method of claim 16, wherein the vantage point of the at least one camera is proximate the vantage point of the user.

19. The method of claim 16, wherein the configuration of the fiducial marker is adapted to present a unique view of the fiducial marker to the at least one camera based on the spatial relationship between the camera and the fiducial marker.

20. The method of claim 16, wherein the fiducial marker is applied to at least one of a guide and a surgical instrument.
